(19) United States
(12) Patent Application Publication
(10) Pub. No.: US 2006/0282080 A1
(43) Pub. Date: Dec. 14, 2006

(54) Vertebral facet stabilizer

(75) Inventors: Todd James Albert, Narberth, PA (US); Rafail Zubok, Midland Park, NJ (US); Mikhail Kvitnitsky, Clifton, NJ (US)

Correspondence Address: KAPLAN GILMAN GIBSON & DERNIER L.L.P., 900 ROUTE 9 NORTH, WOODBRIDGE, NJ, 07095, US

(73) Assignee: Accin Corporation, Clifton, NJ (US)

(21) Appl. No.: 11/443,425

(22) Filed: May 30, 2006

Related U.S. Application Data
(60) Provisional application No. 60/688,421, filed on Jun. 8, 2005.

(51) Int. Cl. A61B 17/70; A61F 2/44

(52) U.S. Cl. 606/61; 623/17.13

(57) ABSTRACT

An vertebral stabilizer includes a spring element defining a longitudinal axis and having first and second ends, each operable to couple to respective first and second bone anchors of a patient, wherein the spring element includes a slanted coil element operable to produce a reaction force in a direction transverse to the longitudinal axis.

### CROSS REFERENCE TO RELATED APPLICATIONS

This application claims the benefit of U.S. Provisional Patent Application No. 60/688,421, filed Jun. 8, 2005, the entire disclosure of which is hereby incorporated by reference.

### BACKGROUND

The present invention generally relates to devices and surgical methods for the treatment of various types of spinal pathologies. More specifically, the present invention is directed to facet stabilization, such as in connection with facet replacement or facet resurfacing.

Back pain is a common human ailment. In fact, approximately 50% of persons who are over 60 years old suffer from lower back pain. Although many incidences of back pain are due to sprains or muscle strains which tend to be self-limited, some back pain is the result of more chronic fibromuscular, osteoarthritic, or ankylosing spondolytic processes of the lumbosacral area. Particularly in the population of over 50 year olds, and most commonly in women, degenerative spine diseases such as degenerative spondylolisthesis (during which one vertebra slides forward over the top of another vertebra) and spinal stenosis (during which the spinal canal markedly—narrows) occurs in a high percentage of the population.

Degenerative changes of the adult spine have traditionally been determined to be the result of the interrelationship of the three joint complex; the disk and the two facet joints. Degenerative changes in the disc lead to arthritic changes in the facet joint and vice versa. One cadaver study of nineteen cadavers with degenerative spondylolisthesis showed that facet degeneration was more advanced than disc degeneration in all but two cases. In mild spondylolisthetic cases, the slip appeared to be primarily the result of predominantly unilateral facet subluxation. Other studies into degenerative changes of the spine have revealed extensive contribution of facet joint degeneration to degenerative spinal pathologies such as degenerative spondylolisthesis, central and lateral stenosis, degenerative scoliosis (i.e., curvature of the spine to one side), and kypho-scoliosis, at all levels of the lumbar spine.

It has been determined that facet joint degeneration particularly contributes to degenerative spinal pathologies in levels of the lumbar spine with sagittally oriented facet joints, i.e. the L4-L5 level.

When intractable pain or other neurologic involvement results from adult degenerative spine diseases, such as the ones described above, surgical procedures may become necessary. Traditionally, the surgical management of disease such as spinal stenosis consisted of decompressive laminectomy alone. Wide decompressive laminectomies remove the entire lamina, and the marginal osteophytes around the facet joint. Degenerative spine disease has been demonstrated to be caused by facet joint degeneration or disease. Thus, this procedure removes unnecessary bone from the lamina and insufficient bone from the facet joint. Furthermore, although patients with one or two levels of spinal stenosis tend to do reasonably well with just a one to two level wide decompressive laminectomy, patients whose spinal stenosis is associated with degenerative spondylolisthesis have not seen good results. Some studies reported a 65% increase in degree of spondylolisthesis in patients treated with wide decompressive laminectomy. The increase in spinal slippage especially increased in patients treated with three or more levels of decompression, particularly in patients with radical laminectomies where all of the facet joints were removed.

To reduce the occurrence of increased spondylolisthesis resulting from decompressive laminectomy, surgeons have been combining laminectomies, particularly in patients with three or more levels of decompression, with multi-level arthrodesis, which surgically fuses the facet joints to eliminate motion between adjacent vertebrae. Although patients who undergo concomitant arthrodesis do demonstrate a significantly better outcome with less chance of further vertebral slippage after laminectomy, arthrodesis poses problems of its own. Aside from the occurrence of further spondylolisthesis in some patients, additional effects include non-unions, slow rate of fusion even with autografts, and significant morbidity at the graft donor site. Furthermore, even if the fusion is successful, joint motion is totally eliminated at the fusion site, creating additional stress on healthy segments of the spine which can lead to disc degeneration, herniation, instability spondylolysis, and facet joint arthritis in the healthy segments.

An alternative to spinal fusion has been the use of invertebral disc prosthesis. Although different designs achieve different levels of success with patients, disc replacement mainly helps patients with injured or diseased discs; disc replacement does not address spine pathologies such as spondylolisthesis and spinal stenosis caused by facet joint degeneration or disease.

While facet replacement or facet resurfacing may address degenerative facet arthrosis, spondylolisthesis and spinal stenosis, it has been discovered that significant improvements may be made by provided additional stabilization of the facet joint.

### SUMMARY OF THE INVENTION

One or more embodiments of the present invention provides a posteriorly disposed system that is designed to stabilize (but not to fuse) the affected vertebral level to alleviate pain stemming from degenerative facet arthrosis, spondylolisthesis and spinal stenosis. Among the functions of some embodiments of the invention is either to replace spinal facet function in connection with a facetechtomy (defined as “facet replacement”) or to work in conjunction with resurfaced facets (defined as “facet supplementation”).

The embodiments of the invention illustrated and described herein permit single level facet replacement and supplementation. It is understood, however, that the system can be applied for multi-level spinal stabilization, where the number of levels is not limited to one, two, three, or more.

The facet replacement and supplementation devices are used single-or bi-laterally (with respect to the spinal process) to augment or substitute spinal facet functions such as providing constraint to the vertebral body within or beyond the biological range of motion and proper disk and soft tissue loading. Various embodiments of the invention can be used with any of the known pedicle screw systems presently utilizing a solid fixation rod of any diameter and are compatible as an integral part of the hybrid multilevel system of spinal fixation. The facet replacement and supplementation devices provide a component of the reactive force in a direction normal to the plane defined by the facet joint by providing a skewed helical spring element in an orientation corresponding to the facet joint angulation. Angulation of the skewed helical-cut or skewed through-cut is oriented such that the cut plane is similar (parallel or acute angle less than 90 degrees) to the plane generated by facets on the instrumented level (“facet plane”).

The reactive force may provide various degrees of rigidity or stiffness to address any physiological condition. The rigidity or stiffness of the device can be achieved through rod geometry (cylinder, hourglass, barrel, etc); rod cross-sectional geometry (rectangular; circular with large or small diameter, etc); cut design and orientation; rod material; elastic inserts between rigid parts; etc.

The skewed helical cut or skewed through cut provides proper anatomical and physiological constraints for vertebral range of motion. The spring element may be offset from the pedicle screws. The offset provides proper orientation of the slots or cuts for restoration of proper kinematics. For example, the orientation of the skewed cut plane should be similar to the plane generated by facets on the instrumented level (facet plane). The offset also provides an increase in the moment arm and minimizes the reaction on the device due to rotation of the spinal column. Embodiments without the offset, but with the skewed helical-cut or skewed through-cut can also be used; however, they will not maximize posterior offset and will require additional care for proper orientation of the cut with respect to the facet plane.

Various embodiments may include different cut orientation methods—markings, special keying or locking features.

The flexibility of one or more embodiments may be enhanced by including an elastic insert either inside the cylindrical section of the rod or between through-cut surfaces.

Other aspects, features, advantages, etc. will become apparent to one skilled in the art when the description of the preferred embodiments of the invention herein is taken in conjunction with the accompanying drawings.

### BRIEF DESCRIPTION OF THE FIGURES

For the purposes of illustrating the invention, there are shown in the drawings forms that are presently preferred, it being understood, however, that the precise arrangements and instrumentalities are not intended to limit the invention.

FIG. 1 is a posterior view of a portion of a spinal column;

FIG. 2 illustrates side views of a spinal column showing facet joint movement;

FIG. 3 is a schematic diagram of a pair of facet joints;

FIG. 4 is a perspective view of an embodiment of a bilateral facet stabilizer in accordance with one or more aspects of the present invention;

FIG. 5 is a rear view of the stabilizer of FIG. 4;

FIG. 6 is a side view of the stabilizer of FIG. 4;

FIG. 7 illustrates side and end views of a conventional helical spring in accordance with the prior art;

FIGS. 8A-C are force diagrams illustrating the physical properties of the spring of FIG. 7;

FIG. 9 is a schematic diagram illustrating the spatial relationships between the turns of the spring of FIG. 7;

FIG. 10 is a schematic diagram illustrating the physical relationship of the turns of a helical spring in accordance with one or more aspects of the present invention;

FIGS. 11A-B illustrate a through-cut spring that includes an offset as is illustrated in the stabilizer of FIG. 4;

FIG. 12 is a schematic diagram illustrating certain functionality provided by the offset feature of the through-cut spring of FIGS. 11A-B;

FIGS. 13A-C are side views of various through-cut springs that may be utilized in the stabilizer of FIG. 4 and or other embodiments herein;

FIGS. 14A-B are perspective views of further alternative embodiments of a spring-like system that may be employed in the stabilizer of FIG. 4 and/or other embodiments herein.

FIG. 15 is a perspective view of an alternative embodiment of a multi-level facet stabilizer in accordance with one or more further aspects of the present invention;

FIGS. 16A-B are perspective and partially cross-sectional views, respectively of a cascaded pair of spring elements that are suitable for use in the facet stabilizer of FIG. 15 and/or other embodiments herein;

FIG. 17 is a perspective view of single one of the spring elements of FIGS. 16A-B, also employing a sleeve element;

FIGS. 18A-B are perspective and partially cross-sectional views, respectively, of the sleeve element of FIG. 17;

FIG. 19 is a perspective view of an alternative embodiment of a multi-level facet stabilizer in accordance with one or more further aspects of the present invention; and

FIGS. 20-21 are perspective views of alternative embodiments of facet stabilizers employing one or more cross link elements in accordance with one or more further aspects of the present invention.

### DETAILED DESCRIPTION

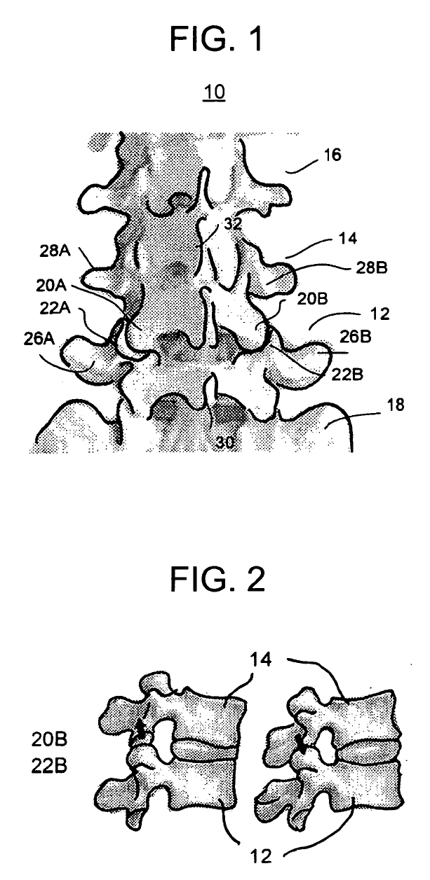

Reference is now made to FIG. 1, which is a posterior view of a portion of a spinal column 10, specifically in the lumbar region. Although the lumbar region of the spine 10 is being discussed herein for illustration, it is understood that the embodiments of the invention are not limited to use in the lumbar region, although that region is preferred. The spinal column 10 includes a plurality of levels, where each level includes a vertebral body 12, 14, 16, etc. The sacrum 18 is partially shown below the various levels of the spinal column 10.

The vertebral body 14 includes superior facet 20A on one side of the spinous process 32 and another superior facet 20B on the other side of the spinous process 32. The vertebral body 14 also includes a pedicle 28A on one side and pedicle 28B on the other side of the spinous process. The next lower vertebral body 12 includes an inferior facet 22A on one side of the spinous process 32 forming a joint with the superior facet 20A, and another inferior facet 22B (on the other side of the spinous process 32) forming a facet joint with the superior facet 20B. The vertebral body 12 also includes pedicles 26A, 26B.

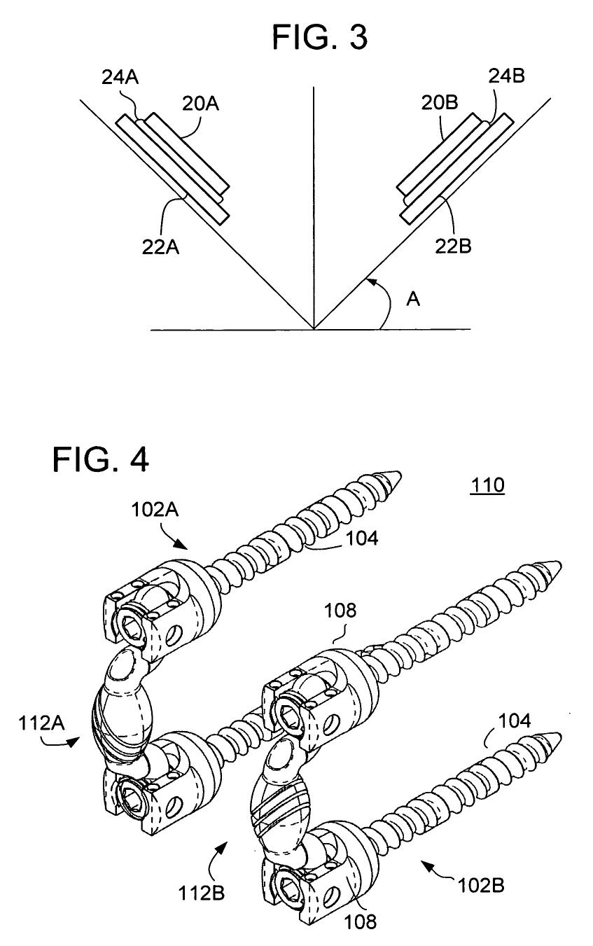

FIG. 2 illustrates side views of the vertebral bodies 12, 14 showing movement of the facet joint produced by the inferior facet 22B and the superior facet 20B. FIG. 3 is a schematic diagram illustrating that the facet joints defined by inferior facets 22A, 22B and superior facets 20A, 20B are angled relative to an axis of the spinal column 10 . FIG. 3 shows that the facet joints are oriented at an angle A from the horizontal, although those skilled in the art will appreciate that the facet joint defines a plane having a compound angle, although for simplicity that compound angle is not shown. In accordance with one or more aspects of the present invention, the angulation of the facet joints is mimicked by the facet stabilizer system discussed below.

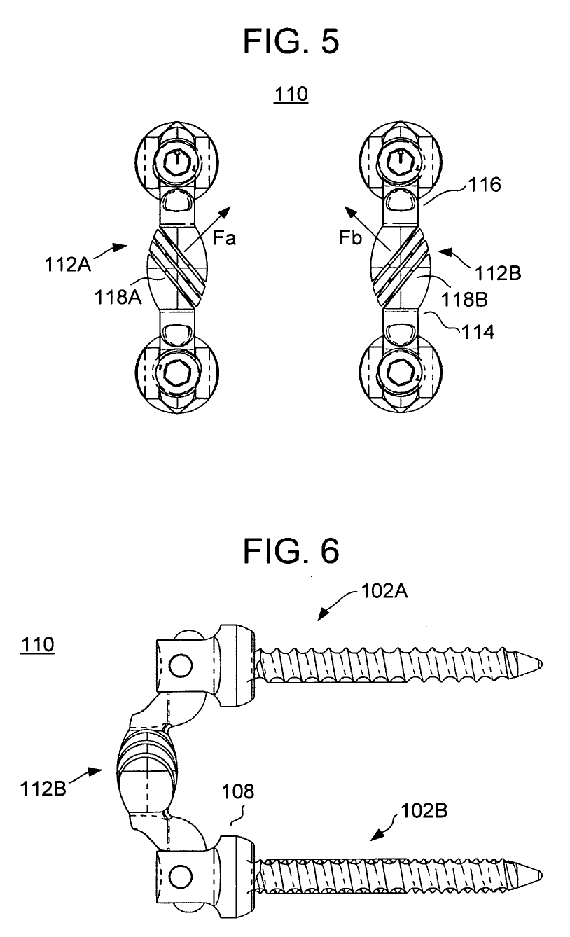

Reference is now made to FIGS. 4, 5, and 6, which illustrate various views of a facet stabilizer system 100 in accordance with one or more embodiments of the present invention. In the illustrated embodiment, a pair of stabilizers 102A, 102B is shown, where each stabilizer 102 may be secured to respective vertebral bones of a patient. For example, the stabilizers 102A, 102B may be bilaterally disposed on respective sides of the spinous processes of the spinal column 10 (FIG. 1). More particularly, each stabilizer 102 includes a pair of bone anchors, such as screws 104, 106, a pair of anchor seats 108, 110, and a spring element 112 (or force restoring member) that cooperate to fix the spring element 112 between adjacent vertebral bones, e.g., bones 12, 14. It is noted that the bone anchors may be implemented in any of the ways available to those skilled in the art, such as the aforementioned screws, as well as glue, bone welding, hooks, cement, etc.

The screws 104, 106 may be pedicle screws that are operable to engage a bore made in the vertebral bone, typically at the pedicles 26, 28. Preferably, the heads of the screws 104, 106 are designed such that the respective anchor seats (or tulips) 108, 110 may articulate with respect to the threaded shaft of the screws 104, 106. It is understood, however, that non-articulating screw and tulip systems (or one-piece systems) may alternatively be employed. Indeed, any of the known or hereafter developed pedicle screws and tulips may be employed to implement the screws 104, 106, and tulips 108, 110 without departing from the spirit and scope of the present invention. For example, it is noted that many of the existing pedicle screw and tulip designs for fixing rods between vertebral bones may be employed to implement this and other embodiments of the present invention.

It is understood that alternative embodiments of the present invention may employ a single stabilizer 102 in a unilateral position (on one side or the other of the spinous processes of adjacent vertebral bones).

The spring elements 112 preferably include a generally longitudinally directed (or extending) body having respective ends 114, 116 for engagement with the screws 104, 106. The spring elements 112 also include a skewed or slanted coil 118 disposed between the ends 114, 116. The skewed or slanted coils 118A, 118B of properly oriented spring elements 112A, 112B preferably mimic the angulation of the facet joints of which they stabilize (or replace). In particular, the skewed coil 118 A is preferably disposed such that it provides a component of the reaction force Fa in a direction substantially normal to a plane defined by the facet joint for which it provides stabilization. Thus, the skewed coil 118A produce the reaction force Fa in a direction transverse to the longitudinal axis of the spring element 112. For example, when the stabilizer 102 A is coupled to vertebral bones 12, 14 on the left side of the spinous processes 30, 32 of the spinal column 10 (FIG. 1), then the orientation of the skewed coil 118A may be disposed in a position to provide a component of the reactive force Fa in a direction normal to a plane defined by the orientations of the superior facet 20A and inferior facet 22A. With reference to FIG. 3, the plane may be parallel to the respective planes of the facets 20A, 22A themselves or the cartilage 24A that is normally between them.

As will be discussed in more detail herein below, the spring characteristics of the skewed coil 118A are preferably such that substantially similar functionality is achieved as compared with the natural anatomy of the facet joint for which stabilization is provided. Among these characteristics is the direction of the reactive force Fa discussed above. Similarly, the skewed coil 118B of a bilaterally disposed system 100 preferably produces a component of the reactive force Fb in a direction that is substantially normal to a plane defined by the opposite facet joint.

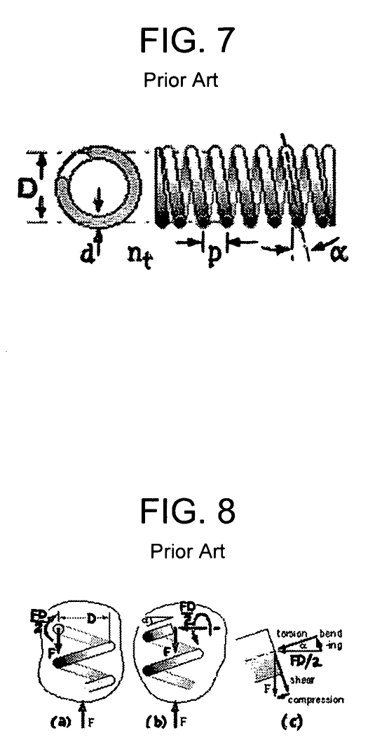

In order to more fully understand that characteristics of the spring element 112 of the stabilizers 102, a brief description of prior art helical springs is now provided with reference to FIGS. 7-10. As discussed, for example, at http://www.mech.uwa.edu.au/DANotes/springs/intro/intro.html, springs are unlike other machine/structure components in that they undergo significant deformation when loaded—their compliance enables them to store readily recoverable mechanical energy. The wire of a helical compression spring as shown in FIG. 7 is loaded mainly in torsion and is therefore usually of circular cross-section. The close-coiled round wire helical compression spring is the type of spring most frequently encountered. It is made from wire of diameter d wound into a helix of mean diameter D, helix angle α, pitch p, and total number of turns nt. This last is the number of wire coils prior to end treatment. In the spring illustrated in FIG. 7, nₜ≈8½.

The ratio of mean coil diameter to wire diameter is known as the spring index, C=D/d.

The free length Lₒ of a compression spring is the spring's maximum length when lying freely prior to assembly into its operating position and hence prior to loading. The solid length Lₛ of a compression spring is its minimum length when the load is sufficiently large to close all the gaps between the coils.

The performance of a spring is characterized by the relationship between the loads (F) applied to it and the deflections (δ) which result, deflections of a compression spring being reckoned from the unloaded free length as shown in the animation.

The F-δ characteristic is approximately linear provided the spring is close-coiled and the material elastic. The slope of the characteristic is known as the stiffness of the spring k =F/δ (also known as spring “constant,” “rate,” “scale,” or “gradient”) and is determined by the spring geometry and modulus of rigidity as will be shown.

The free body FIG. 8(a) of the lower end of a spring whose mean diameter is D: embraces the known upward load F applied externally and axially to the end coil of the spring; and cuts the wire transversely at a location which is remote from the irregularities associated with the end coil and where the stress resultant consists of an equilibrating force F and an equilibrating rotational moment FD/2.

The wire axis is inclined at the helix angle a at the free body boundary in the side view, FIG. 8(b) (note that this is first angle projection). An enlarged view of the wire cut conceptually at this boundary FIG. 8(c) shows the force and moment triangles from which it is evident that the stress resultant on this cross-section comprises four components—a shear force (F cosα), a compressive force (F sinα), a torque (½ FD cosα)and a bending moment (½ FD sinα).

Assuming the helix inclination a to be small for close-coiled springs—then sinα≈0, cosα≈1, and the significant loading reduces to torsion plus direct shear. The maximum shear stress at the inside of the coil will be the sum of these two component shears:

$$\begin{matrix}τ = {τ}_{torsion} + {τ}_{direct} \\ = Tr / J + F / A \\ = ( FD / 2 ) ( d / 2 ) / ( π {d}^{4} / 32 ) + F / ( π {d}^{2} / 4 ) \\ = ( 1 + 0.5 d / D ) 8 FD / π {d}^{3}\end{matrix} \\ τ = K 8 FC / π {d}^{2} \qquad ( 1 )$$

The stress factor, K, assumes one of three values, either: K=1 when torsional stresses only are significant—ie. the spring behaves essentially as a torsion bar; K=Kₛ≡1+0.5/C which accounts approximately for the relatively small direct shear component noted above, and is used in static applications where the effects of stress concentration can be neglected; or K=Kₕ≈(C+0.6)/(C−0.67), which accounts for direct shear and also the effect of curvature-induced stress concentration on the inside of the coil (similar to that in curved beams). Kₕ should be used in fatigue applications; it is an approximation for the Henrici factor, which follows from a more complex elastic analysis as reported in Wahl op cit. It is often approximated by the Wahl factor K_{w}=(4C−1)/(4C−4)+0.615/C. The factors increase with decreasing index:

The deflection δ of the load F follows from Castigliano's theorem. Neglecting small direct shear effects in the presence of torsion:

$$\begin{matrix}δ = \partial U / \partial F \\ = \partial / \partial F [ {\int }_{length} ( {T}^{2} / 2 GJ ) ⅆ s ] ,\end{matrix}$$

$$where$$

$$\begin{matrix}T = FD / 2 \\ = {\int }_{length} ( T / GJ ) ( \partial T / \partial F ) ⅆ s \\ = ( T / GJ ) ( D / 2 ) * ( wire length ) \\ = ( FD / 2 GJ ) ( D / 2 ) {n}_{a} π D ,\end{matrix}$$

which leads to:
k=F/δ=Gd/8nₐC³, (2)
in which nₐ is the number of active coils (Table 1)

Despite many simplifying assumptions, equation (2) tallies well with the experiment provided that the correct value of rigidity modulus is incorporated, e.g., G=79GPa for cold drawn carbon steel.

Standard tolerance on wire diameters less than 0.8mm is 0.01 mm, so the error of theoretical predictions for springs with small wires can be large due to the high exponents which appear in the equations. It must be appreciated also that flexible components such as springs cannot be manufactured to the tight tolerances normally associated with rigid components. The spring designer must allow for these peculiarities. Variations in length and number of active turns can be expected, so critical springs are often specified with a tolerance on stiffness rather than on coil diameter. The reader is referred to BS 1726 or AE-11 for practical advice on tolerances.

Compression springs are no different from other members subject to compression in that they will buckle if the deflection (i.e., the load) exceeds some critical value δ_{crit}which depends upon the slenderness ratio Lₒ/D rather like Euler buckling of columns, thus:
C₁δ_{crit}/Lₒ=1−√[1−(C₂D/λLₒ)²], (3a)
in which the constants are defined as follows:
c₁=(1+2ν/(1+ν)=1.23 for steel; and
c₂=Π√[(1+2ν/(2+ν)]=2.62 for steel.

The end support parameter λreflects the method of support. If both ends are guided axially but are free to rotate (like a hinged column) then λ=1. If both ends are guided and prevented from rotating then λ=0.5. Other cases are covered in the literature. The plot of the critical deflection is very similar to that for Euler columns.

A rearrangement of (3a) suitable for evaluating the critical free length for a given deflection is:
L_{o.crit}=[1+(c₂D/c₁λδ)²]c₁δ/2 (3b)

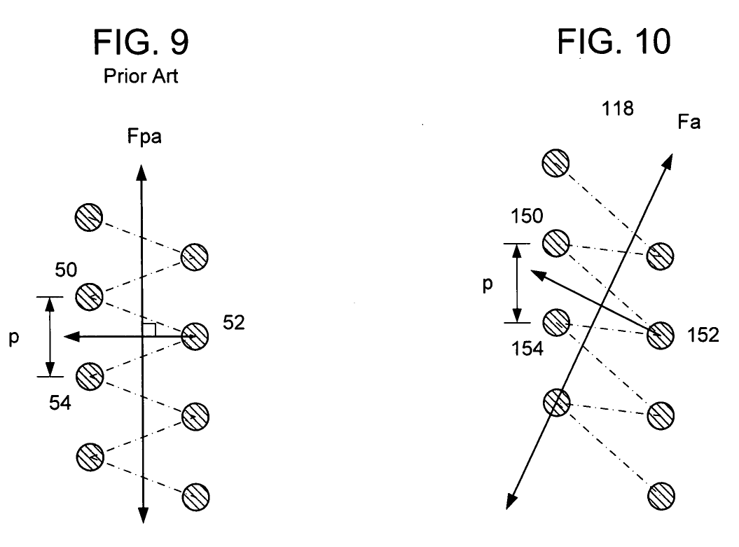

With reference to FIGS. 9 and 10 and the discussion above, it will be evident to those of skill in the art that a standard prior art helical spring cannot provide the desired reaction force Fa, Fb as is produced by the spring elements 112 of the stabilizers 102. In particular, as is shown in FIG. 9, the cross-sectional positions of the turns of a standard helical spring are designed to provide a force in the direction shown by the arrow Fpa. In particular, a given turn of the prior art helical spring will result in cross-sectional profiles 50, 52, and 54 being positioned such that the cross-sectional profile 52 bisects the pitch, p, between the other two cross-sectional profiles 50, 54. This may be demonstrated for every active turn of the spring. Thus, the force Fpa is perpendicular to the plane passing through the cross-sectional profile 52. Notably, the force Fpa cannot be oriented to mimic the functionality of a facet joint of the spinal column 10. Indeed, if the prior art spring of FIG. 9 were loaded in a traversed direction (as would be the case in stabilizing a facet joint), then the prior art spring would buckle and potentially cause further complications in a patient.

The skewed coil 118 of FIG. 10 provides a very different reactive force Fa, which is transverse to the longitudinal orientation of the turns of the skewed coil 118 (e.g., the turns follow the longitudinally extending body). Notably, the cross-sectional profiles 150, 152, 154 of a given turn of the skewed coil 118 are not positioned as in the prior art. Rather, the cross-sectional profile 152 is skewed downward (or upward in alternative embodiments) from the bisecting position such that the force Fa (again perpendicular to the plane passing through the cross-sectional profile 152 and the bisecting position) is transversely oriented. Advantageously, this functionality enables the longitudinally directed spring element 112 to provide a reaction force F in a transverse direction with respect to the longitudinal axis of the spring element 112.

The above-described structure and function of the spring elements 112A, 112B result in at least the following characteristics: (i) the slanted coils 18A, 118B may be slanted at least partially toward one another; (ii) at least one vector component of each the reaction forces Fa, Fb is at least parallel to (and potentially co-axial with)the longitudinal axes of the spring elements 112A, 112B, respectively; (iii) at least one vector component of each the reaction forces Fa, Fb is at least transverse to the longitudinal axes of the spring elements 112A, 112B, respectively; (iv) and at least one vector component of each the reaction forces Fa, Fb are at least parallel to (and potentially co-axial with) one another.

Further, the articulation of the respective tulips 108, 110 and the rotatability of the ends 114, 116 that engage same permit adjustability of the reaction force F such that it may be directed in a position substantially normal to the facet joint for which stabilization is provided or for which facet replacement has been made.

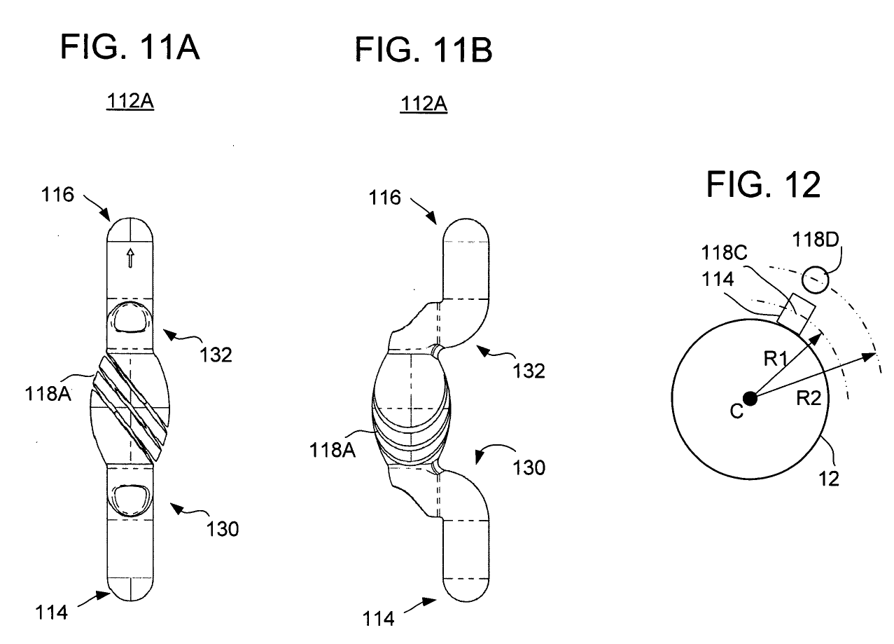

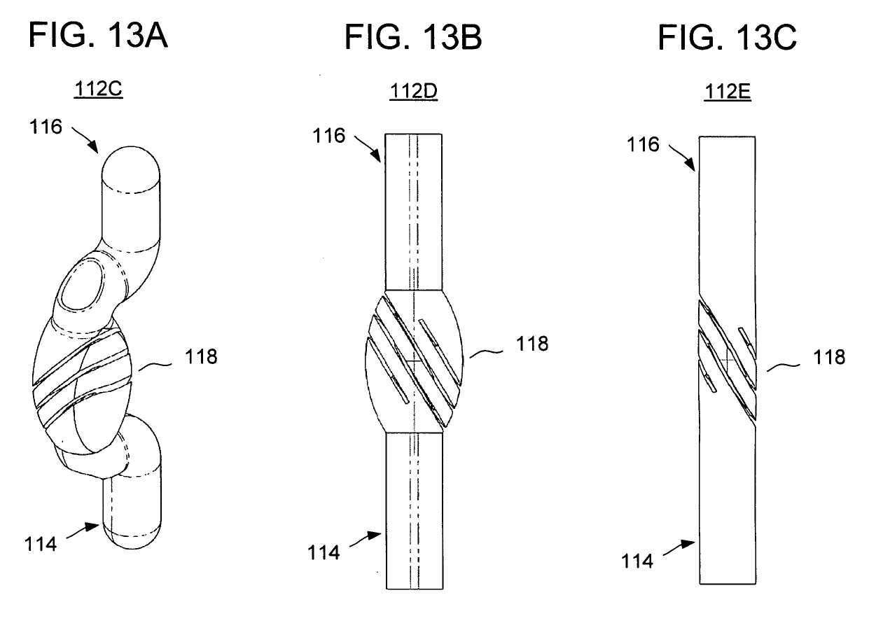

As is depicted in FIGS. 11A, 11B, and 12, the spring element 112 may include respective offsets 130, 132, which place the skewed coil 118 outside the axis of orientation (e.g., a longitudinal axis) in which the respective ends 114, 116 are disposed. By contrast, with reference to FIGS. 13B, 13C, when a spring element 112D or 112E is employed, the skewed coil 118C is substantially in line with the respective tulips 108, 110. Therefore, the radius R1 from a center C of, for example, the vertebral bone 12 to a center of the skewed coil 118C establishes the moment arm and resulting stiffness required to implement the stabilizer 102. When the spring element 112A is implemented utilizing the embodiment illustrated in FIGS. 11A, 11B, however, a radius of R2 (which is greater than R1) is achieved and a greater moment arm is advantageously enjoyed by the skewed coil 118D. Thus, the skewed coil 118D need not be as stiff and as strong as the skewed coil 118C. Lesser demands on stiffness and strength of the device result in less bulky and less invasive construct. Thus, different materials and/or spring characteristics and dimensions may be employed depending on whether an offset is employed or not.

As can be seen in FIGS. 11A, 11B, the skewed coil 118 may take on a barrel shape when viewed transversely to the longitudinal axis or plane extending from end 114 to end 116. This shape provides an increase in the diameter D of the turns of the coil and a resultant increase in the stiffness of the spring action without increase of critical device dimensions. It is noted that other configurations are contemplated by the present invention, including cylindrical configurations (e.g., an in-line configuration, FIG. 13C), hourglass shapes, the barrel shape, and other complex geometries. Further, the general shape of the spring element 112 may be of circular cross-section, rectangular cross-section, and other complex geometries as within the purview of one of ordinary skill in the art having considered this specification. For example, FIG. 13A illustrates a combined barrel shape and rectangular shape, which is useful in reducing the overall width of the spring element 112 (e.g., to be the same width as the ends 114, 116 ), yet retaining at least some of the increased stiffness of a barrel shaped spring element 112. Thus, the spring element 112 is at least partially barrel-shaped when viewed in at least one plane, and substantially rectangular shaped when viewed in at least one other plane.

The skewed coil 118 of the various embodiments of the present invention may be implemented utilizing a helical coil of the type illustrated in FIG. 10, where the skew takes the cross-sectional profile 152 off center in one direction or the other by any amount. Alternatively, the skewed coil 118 may be implemented by way of a series of through-cuts into a hollow rod as is illustrated in FIGS. 11A, 11B, and 13A-C. Those skilled in the art will appreciate that the through-cut embodiments of the present invention exhibit substantially similar cross-sectional profiles as illustrated in FIG. 10.

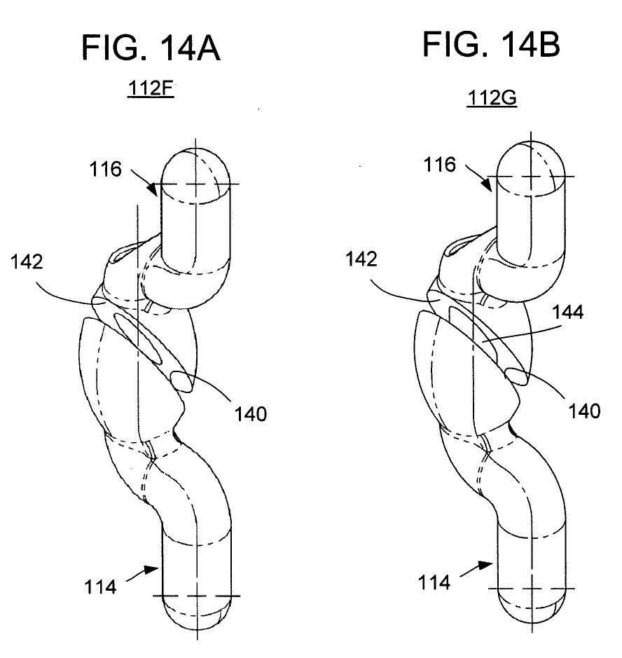

With reference to FIGS. 14A-B, the spring element 112F, 112G may be implemented by way of a pair of angularly spaced-apart surfaces 140, 142. In other words, the surfaces 140, 142 are slanted with respect to the longitudinal axes of the elements 112F, 112G. In one or more embodiments, the bearing surfaces 140, 142 are substantially parallel to one another. Alternatively or additionally, the bearing surfaces 140, 142 are slidingly engageable with one another such that they mimic anatomical motion of superior and inferior facets of a facet joint. Additionally or alternatively, a resilient material 144, such as a polymeric material, may be disposed between the surfaces 140, 142 (FIG. 14B). The spring characteristics of the surfaces 140, 142 may thus be adjusted from no resiliency to the resilient properties of the material 144. Notably, the spring elements 112F, 112G are shown having the offset feature discussed hereinabove. In alternative embodiments, the offset feature may be omitted in favor a substantially in-line configuration.

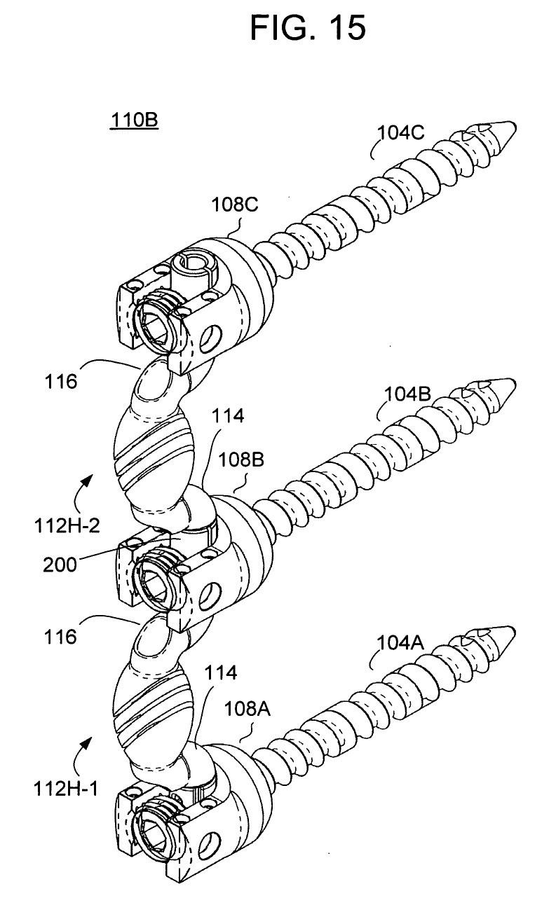

It noted that a single stage stabilizer system 100 has been illustrated and discussed above. It is contemplated, however, that multi-stage systems may be implemented by cascading additional levels of the stabilizers 102, as is shown in FIG. 15, such that additional levels of the spinal column 10 may be stabilized as may be desired by the surgeon. As illustrated, the vertebral stabilizer 110B includes at least first, second, and third bone anchors 104A-C, each for coupling to a respective vertebral bone of a patient. The vertebral stabilizer 110B also includes at least first and second spring elements 112H-1, 112H-2, each having ends 114, 116 defining respective longitudinal axes. Each of the spring elements 112H-1, 112H-2 are coupled to a pair of the bone anchors 104 such that they are in substantial longitudinal axial alignment. Thus, the end 114 of the first spring element 112H-1 is coupled to the bone anchor 104A, the end 116 of the first spring element 112H-1 and the end 114 of the second spring element 112H-2 are coupled to the bone anchor 104B, and the end 116 of the second spring element 112H-2 is coupled to the bone anchor 104C.

While the illustrated vertebral stabilizer 110B is a two-level system, those skilled in the art will appreciate from the description herein that the number of levels may be increased as desired by cascading additional spring elements together.

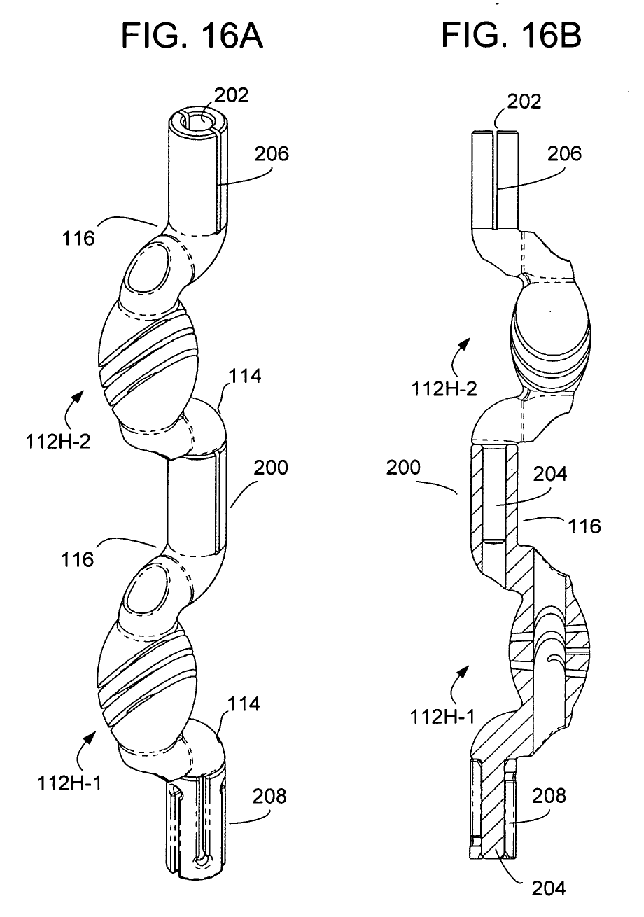

In this regard, the vertebral stabilizer 110B further includes a coupling element 200 operable to join the end 116 of the first spring element 112H-1 to the end 114 of the second spring element 112H-2. Although any number of mechanical implementations may be employed to form the coupling element 200, one example is best seen in FIGS. 16A-B and 17. The coupling feature 200 includes a bore 202 disposed at at least one end (for example, end 116 ) of one of the spring elements 112H-1, and a shaft 204 disposed at at least one end (for example, end 114 ) of the spring element 112H-2. The bore 202 and the shaft 204 are sized and shaped such that the shaft 204 may slide into the bore 202 to couple the ends 114, 116 of the first and second spring elements 112H-1, 112H-2 together.

The bore 202 may be slotted by way of one or more slots 206 such that a compressive force thereon causes a diameter of the bore 202 to reduce, and interior surfaces of the bore 202 to be urged against the shaft 204 to fix the ends 114, 116 of the first and second spring elements together 112H-1, 112H-2. Thus, the coupling element 200 is operable to fix the ends 114, 116 of the first and second spring elements 112H-1, 112H-2 together in response to pressure applied thereto when coupled to the bone anchors 104, e.g., by way of tightening the tulip 108 thereof.

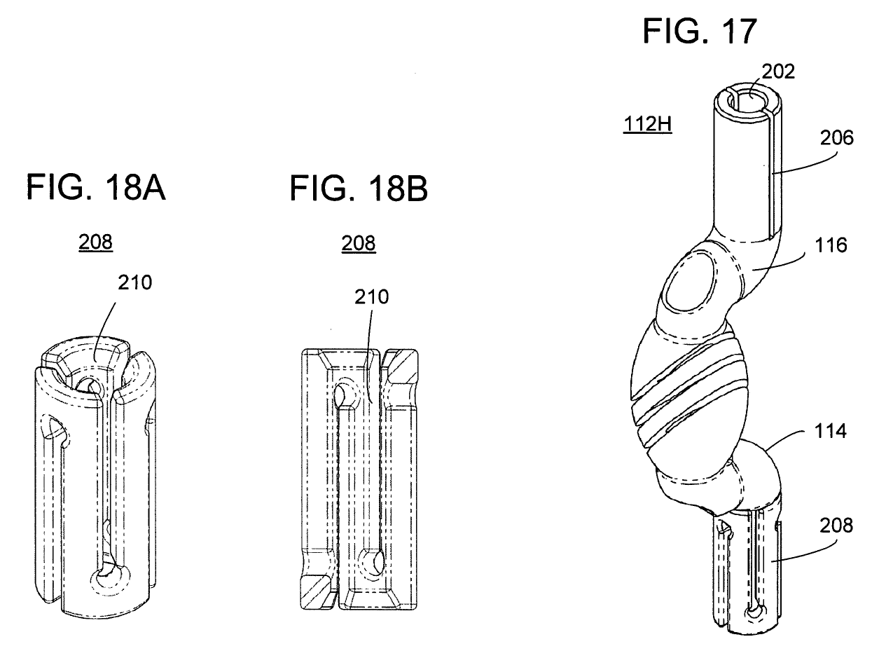

It is noted that any un-mated shaft 204 may be treated using a sleeve 208 including a bore 210 that is sized and shaped to receive the shaft 204. It is preferred that the sleeve 210 is sized and shaped to complement one or more cross-sectional dimensions (e.g., the diameter) of the shaft 204 to substantially match one or more cross-sectional dimensions (e.g., the diameter) of the end 114 to which it is attached. The sleeve 208 may include at least one slot 212 extending from the bore 210 to a surface of the sleeve 208 such that a compressive force about the sleeve 208 causes a diameter of the bore 210 to reduce. The sleeve 208 may be employed in a single level configuration as is illustrated in FIG. 17.

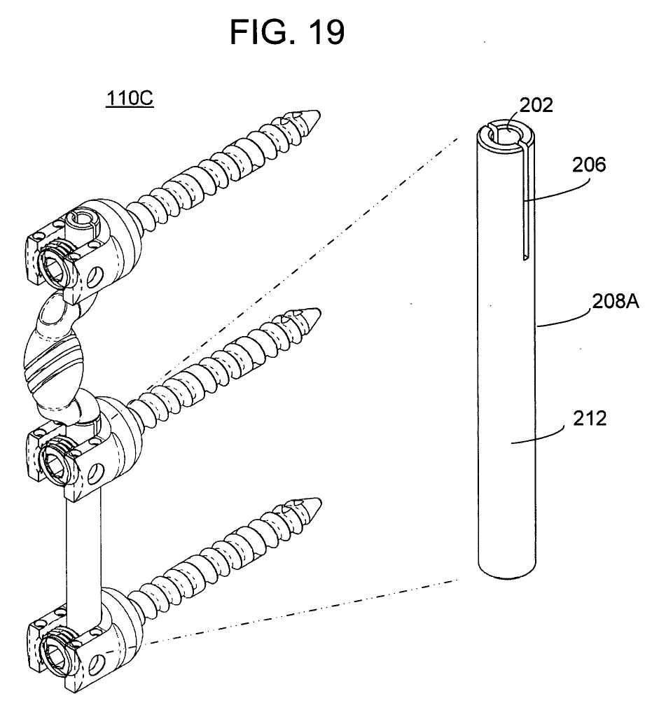

As is illustrated in FIGS. 15-18, the sleeve 208 may be of substantially the same length as the shaft 204. Alternatively, the sleeve 208 may be sized to have a length longer than the shaft 204. For example, as illustrated in FIG. 19, an alternative embodiment vertebral stabilizer 110C includes a sleeve 208 A having a substantially rigid section 212 extending longitudinally away from the bore 202. This has utility in multi-level applications.

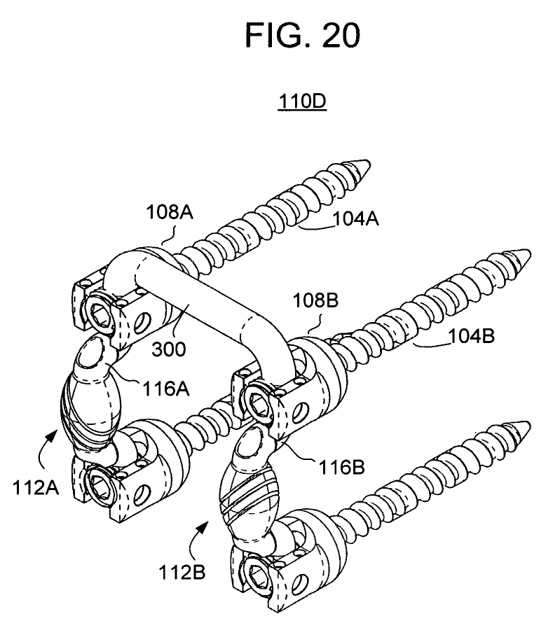

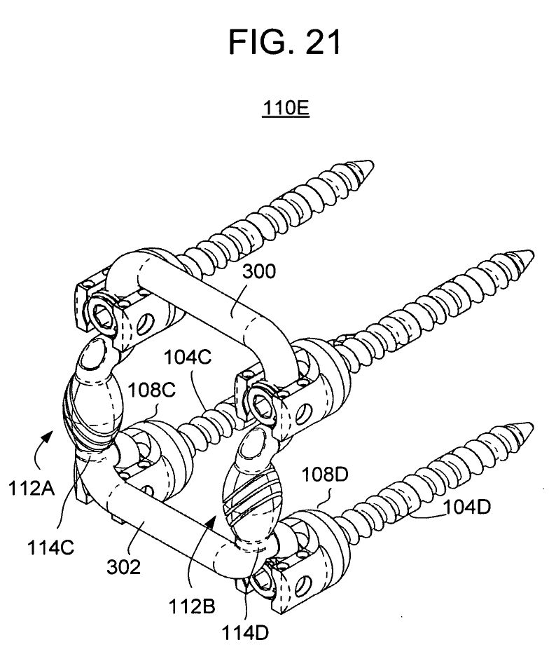

With reference to FIGS. 20-21, one or more embodiments of the present invention may employ alternative vertebral stabilizer systems 110D, 110E. In the vertebral stabilizer system 110D illustrated in FIG. 20, a cross link element 300 may be employed to couple adjacent bone anchors 104A, 104B together. In this embodiment, the cross link element 300 is operable to engage the respective ends 116A, 116B of the spring elements 112A, 112B through the tulips 108A, 108B. Although any number of mechanical implementations may be employed to couple the cross link element 300 to the respective ends 116A, 116B of the spring elements 112A, 112B, one such approach is the bore/shaft coupling 200 discussed above with respect to the multi-level embodiment (FIGS. 15-19 ).

In the vertebral stabilizer system 110E illustrated in FIG. 21, a cross link element 302 may be alternatively or additionally employed to couple the other adjacent bone anchors 104C, 104D together. In this embodiment, the cross link element 302 is operable to engage the respective ends 114C, 114D of the spring elements 112A, 112B without implicating the tulips 108C, 108D.

Among the aspects and functionality of one or more of the embodiments of the invention are:
Replacement or augmentation of spinal facet function in the event of a facetechtomy or a resurfaced or machined facets (facet supplementation).
The skewed helical-cut or skewed through-cut provides proper anatomical and physiological constraints for vertebral range of motion.
Posterior disc collapse is inhibited with the minimal restriction of the vertebral body biological ROM.
Minimum pre-determined distance between bone anchors (or any attachment points) without limiting any motion (displacement, rotation, subluxation, flexion, extension, bending or any combination thereof) is maintained.
Any screw system presently used for solid rod fixation may be employed to attach the system.
Single level or multilevel stabilization may be achieved.
System flexibility and stiffness may be controlled.
Offset feature may maximize posterior offset and minimize reaction on the device.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A vertebral stabilizer, comprising:
first and second bone anchors each for coupling to a respective vertebral bone of a patient; and
a spring element having first and second ends defining a longitudinal axis, each coupled to a respective one of the first and second bone anchors,
wherein the spring element includes a slanted coil element operable to produce a reaction force in a direction transverse to the longitudinal axis.

2. The vertebral stabilizer of claim 1, wherein at least a component of the reaction force is in a direction normal to a plane defined by a facet joint, which includes adjacent superior and inferior facets of first and second vertebral bones to which the respective bone anchors are coupled.

3. The vertebral stabilizer of claim 1, wherein a bisecting cross-section of at least one turn of the slanted coil element includes three cross-sectional profiles, two of which are in longitudinal alignment and define a pitch of the slanted coil element, and the third of which is longitudinally offset from a midpoint between the two cross-sectional profiles.

4. The vertebral stabilizer of claim 1, wherein a plurality of turns of the slanted coil element are formed from one or more helical coils.

5. The vertebral stabilizer of claim 1, wherein the spring element includes a hollow interior volume; and
a plurality of turns of the slanted coil element are formed from one or more through-cuts from an exterior surface to the interior volume of the slanted coil element.

6. The vertebral stabilizer of claim 1, wherein the spring element is substantially barrel-shaped when viewed longitudinally.

7. The vertebral stabilizer of claim 1, wherein the spring element is at least one of substantially barrel-shaped, cylindrically shaped, at least partially spherically shaped, and hourglass shaped, when viewed longitudinally.

8. The vertebral stabilizer of claim 1, wherein the spring element is at least partially barrel-shaped when viewed in at least one plane and substantially rectangular shaped when viewed in at least one other plane.

9. The vertebral stabilizer of claim 1, wherein:
the first and second ends of the spring element define a first longitudinal axis; and
the slanted coil element defines a second longitudinal axis, which is not axially aligned with the first longitudinal axis.

10. The vertebral stabilizer of claim 9, wherein the second longitudinal axis is laterally offset from the first longitudinal axis.

11. A vertebral facet stabilizer, comprising:
a spring element having first and second ends, each operable to couple to respective first and second bone anchors,
wherein the spring element includes a slanted coil element operable to produce at least a component of a reaction force in a direction normal to a plane defined by a facet joint, which includes adjacent superior and inferior facets of first and second vertebral bones of a patient.

12. An vertebral facet stabilizer, comprising:
a spring element having first and second ends, each operable to couple to respective first and second bone anchors, and a slanted coil element,
wherein a bisecting cross-section of at least one turn of the slanted coil element includes three cross-sectional profiles, two of which are in longitudinal alignment and define a pitch of the slanted coil element, and the third of which is longitudinally offset from a midpoint between the two cross-sectional profiles.

13. An interconnecting member for use in a vertebral stabilizer, comprising:
a spring element disposed defining a longitudinal axis, wherein the spring element includes a slanted coil
element operable to produce a reaction force in a direction transverse to the longitudinal axis.

14. A vertebral stabilizer, comprising:
a first spring element having first and second ends, each operable to couple to respective first and second bone anchors, the first spring element defining a first longitudinal axis and including a slanted coil element operable to produce a first reaction force in a direction transverse to the first longitudinal axis;
third and fourth bone anchors for coupling to the respective vertebral bones;
a second spring element having first and second ends, each coupled to respective third and fourth bone anchors, the second spring element defining a second longitudinal axis including a slanted coil element operable to produce a second reaction force in a direction transverse to the second longitudinal axis,
wherein the first and second spring elements are coupled bi-laterally to the respective vertebral bones.

15. The vertebral stabilizer of claim 14, wherein the first and second slanted coils are slanted at least partially toward one another.

16. The vertebral stabilizer of claim 14, wherein at least one vector component of each the first and second reaction forces is at least parallel to the first and second longitudinal axes, respectively.

17. The vertebral stabilizer of claim 14, wherein at least one vector component of each the first and second reaction forces is at least transverse to the first and second longitudinal axes, respectively.

18. The vertebral stabilizer of claim 14, wherein at least one vector component of each the first and second reaction forces are at least parallel to one another.

19. The vertebral stabilizer of claim 14, further comprising a first cross link element operable to couple the first and third bone anchors to one another.

20. The vertebral stabilizer of claim 14, further comprising a second cross link element operable to couple the second and fourth bone anchors to one another.

21. A vertebral facet stabilizer, comprising:
an interconnecting element having first and second ends, each operable to couple to respective first and second bone anchors,
wherein the interconnecting element includes first and second bearing surfaces defining a longitudinal axis, being disposed between the first and second ends, and being substantially slanted with respect to the longitudinal axis.

22. The vertebral facet stabilizer of claim 21, wherein the first and second bearing surfaces are substantially parallel to one another.

23. The vertebral facet stabilizer of claim 22, wherein the first and second bearing surfaces are slidingly engageable with one another such that they mimic anatomical motion of superior and inferior facets of a facet joint.

24. The vertebral facet stabilizer of claim 22, further comprising a resilient element disposed between the first and second bearing surfaces.

25. A vertebral stabilizer, comprising:
at least first and second spring elements, each having first and second ends, each defining first and second longitudinal axes, respectively, and each operable to couple to a pair of bone anchors from among at least first, second, and third bone anchors, the bone anchors for coupling to respective vertebral bones of a patient, and the first and second spring elements coupling such that they are in substantial longitudinal axial alignment,
wherein the first and second spring elements each include a slanted coil element operable to produce first and second reaction forces, respectively, in first and second directions, respectively, that are transverse to the first and second longitudinal axes.

26. The vertebral stabilizer of claim 25, further comprising a coupling feature operable to join one of the first and second ends of the first spring element to one of the first and second ends of the second spring element.

27. The vertebral stabilizer of claim 26, wherein:
the coupling feature includes a bore disposed at the one end of the first spring element, and a shaft disposed at the one end of the second spring element; and
the bore and shaft are sized and shaped such that the shaft may slidingly enter the bore to couple the ends of the first and second spring elements together.

28. The vertebral stabilizer of claim 27, wherein the bore is slotted such that a compressive force causes a diameter of the bore to reduce and interior surfaces of the bore may be urged against the shaft to fix the ends of the first and second spring elements together.

29. The vertebral stabilizer of claim 26, wherein the coupling feature is operable to fix the ends of the first and second spring elements together in response to pressure applied thereto when coupled to one of the bone anchors.

30. The vertebral stabilizer of claim 25, wherein the slanted coil elements of the first and second spring elements are slanted substantially in the same direction.

31. The vertebral stabilizer of claim 30, wherein at least one of:
at least one vector component of each the first and second reaction forces is at least parallel to the first and second longitudinal axes, respectively;
at least one vector component of each the first and second reaction forces is at least transverse to the first and second longitudinal axes, respectively; and
the first and second reaction forces are substantially parallel to one another.

32. An interconnecting member for use in a vertebral stabilizer, comprising:
first and second ends operable for connection to respective bone anchors;
a spring element disposed between the first and second ends defining a longitudinal axis; and
at least one of: (i) a bore disposed at one of the first and second ends of the spring element, and (ii) a shaft disposed at the other of the first and second ends of the spring element, wherein
the spring element includes a slanted coil element operable to produce a reaction force in a direction transverse to the longitudinal axis.

33. The interconnecting member of claim 32, wherein the bore and shaft are sized and shaped such that at least one of: (i) the shaft may slidingly enter a substantially similar bore of a further interconnecting member, and (ii) the bore may slidingly receive a substantially similar shaft of a further interconnecting member, to couple the interconnecting member to the further interconnecting member.

34. The interconnecting member of claim 33, wherein the bore is slotted such that a compressive force causes a diameter of the bore to reduce and interior surfaces of the bore to be urged against the shaft of the further interconnecting member.

35. The interconnecting member of claim 33, further comprising a sleeve including a second bore sized and shaped to receive the shaft and at least one slot extending from the second bore to a surface of the sleeve such that a compressive force about the sleeve causes a diameter of the second bore to reduce.

36. The interconnecting member of claim 35, wherein the sleeve is sized and shaped to complement one or more cross-sectional dimensions of the shaft to substantially match those of the other of the first and second ends of the spring element.

37. The interconnecting member of claim 35, wherein a length of the sleeve is substantially the same as a length of the shaft.

38. The interconnecting member of claim 35, wherein a length of the sleeve is longer than a length of the shaft.

39. The interconnecting member of claim 38, wherein the sleeve includes a substantially rigid section extending longitudinally away from the second bore.
